(19) United States
(12) Patent Application Publication
(10) Pub. No.: US 2005/0176811 A1
(43) Pub. Date: Aug. 11, 2005

(54) Vitamin combination for providing protection during the chemotherapy and/or radio-therapy of malignant tumours

(76) Inventors: Tatjana Sergejevna Morozkina, Minsk (BY); Vladimir Nikolajevich Sukolinski, Minsk (BY)

Correspondence Address: ROTHWELL, FIGG, ERNST & MANBECK, P.C., 1425 K STREET, N.W., SUITE 800, WASHINGTON, DC, 20005, US

(21) Appl. No.: 10/510,750

(22) PCT Filed: Mar. 27, 2003
(86) PCT No.: PCT/EP03/03205

(30) Foreign Application Priority Data
Apr. 10, 2002 (DE) 102 15 765.0

(51) Int. Cl. A61K 31/375; A61K 31/355; A61K 31/07

(52) U.S. Cl. 514/458; 514/474; 514/725

(57) ABSTRACT

The invention relates to the use of a vitamin combination for improving the tolerance to chemotherapy and/or radiotherapy of malignant tumours.

The present invention concerns the use of an agent for improving the tolerability of the chemotherapy or/and radiation therapy of malignant tumours.

The occurrence of malignant tumours has steadily increased in the last 20 years. Studies on patients with stomach cancer in various stages has shown that a surgical removal of the malignant tumour is expedient in patients with stomach cancer at an early stage but that surgical treatment alone is ineffective when the cancer has reached a more advanced stage. Local recurrences and distant metastases occur in 20 to 30% of such patients in the first year after the operation and the percentage is already 40% within the second year of monitoring. The average 5 year survival rate of patients with colon cancer that had been treated surgically is about 50%. Most of the monitored patients had local recurrences and distant metastases. It turned out that antitumour therapies such as chemotherapy or polychemotherapy or/and radiation therapy are necessary in addition to surgical treatment.

In radiation therapy ionizing radiation is used with the aim of damaging the tumour tissue. Ionizing radiation (e.g. gamma or X-ray radiation) can, however, also damage healthy tissue by generating radicals. Severe radiation complications usually occur at effective radiation doses and hence radiation therapy is normally carried out at considerably lower doses than would be required for the therapy to have an optimal effect. Radiation dermatitis, pulmonary fibrosis, damage to the mucous membranes in the gastrointestinal tract, increased pain and immunodepression for example occur as radiation complications. Since the radiation also attacks healthy tissue, the success of the radiation therapy is considerably reduced which results in a reduced life expectancy of the affected patients. Studies have shown for example that in patients with tumours at an advanced stage, doses of 60 Gy are necessary to have a good effect. However, such high doses are unacceptable without protecting the organism due to the considerable side-effects. Information on the results of radiation therapies are given in Jarmonenco (“Radiobiologie: Menschen und Tiere”). The following Table 1 which is taken from this citation shows the tolerance dose for ionizing radiation for vital organs.

TABLE 1

| | | | max. | |
|---|---|---|---|---|
| | maximal SFD | | isoequivalency | tolerance dose |
| organ | (Gy) | FTD | (Gy) | (Gy) |
| skin | 7.2 | 119 | 72 | 50-70 |
| spinal cord | 3.3 | 35.5 | 21 | 50-57 |
| left lobule of | 7.1 | 115.6 | 70 | 30-50 |
| the liver | | | | |
| left kidney | 6.7 | 107 | 64 | 13-25 |
| pancreas: | | | | |
| head | 6.9 | 111 | 67 | — |
| body | 5.4 | 76 | 46 | 30-100 |
| tail | 2.5 | 23 | 14 | — |
| small intestine | 5.4 | 76 | 46 | 35-45 |

| | |
|---|---|
| maximum single focal | Absorbed dose for the organs stated in the table |
| dose (SFD): | after each radiation treatment (5 treatments) |
| | with an SFD of 7 Gy. |
| factor-time dose (FTD): | In order to recalculate the damaging effect in |
| | the tissue after irradiation with higher fractions |
| | (7 Gy) compared to the standard methods |
| | (SFD = 2 Gy). |
| max. isoequivalency: | Total absorbed dose for various organs after 5 |
| | radiation treatments (calculated according to |
| | special radiological tables FTD). |
| tolerance dose: | Maximum tolerated radiation dosage for |
| | various organs without radiation protective |
| | agents. |

An oxidation process that can be stimulated by irradiation is for example liquid peroxidation in which free radicals lead to a decomposition of lipids. Lipid peroxidation products are formed in this process such as malonic dialdehyde (MDA) and 4-hydroxy-2-trans-hexenal. Lipid peroxidation products are usually mutagenic substances that can damage the organism. The oxidation in the body that is induced by irradiation can result in the destruction of healthy tissue, DNA and cell membranes.

Previously agents such as mexamine and cysteamine hydrochloride were used to protect healthy tissue during radiation therapy. The radical trapping properties of cysteamine (2-aminoethanethiol) have led to its use to treat radiation damage. However, these conventional radiation protective agents have a high toxicity which results in considerable side-effects. The doses of radiation protective agents that have to be administered to carry out an effective radiation therapy would be unacceptable for health reasons. Thus their range of applications and duration of their application is extremely limited.

Cell poisons are administered in chemotherapy and polychemotherapy which are intended to specifically interfere with the growth of tumour tissue. 5-Fluorouracil (5-Fu), cis-platinum and doxorubicin are traditionally used as cytostatic agents. However, these agents have a low selectivity for the tumour tissue and thus damage healthy tissue to a considerable extent. The cytostatic agents that are generally used can initiate lipid peroxidation due to the occurrence of free radicals in the organism. Hence the side-effects of chemotherapy that occur limit the dose of these agents that can be used and thus the effectiveness of the antitumour therapy.

As is the case for single therapies, damage to healthy regions of the organism also occurs in the case of a combined radiation/chemotherapy. Even when tumour patients are only surgically treated, the already weakened organism of the patients is subjected to additional stress as a result of which oxidation processes can for example be stimulated in the body.

Hence a major problem in treating malignant tumours by surgical interventions or/and chemo- or/and radiation therapy is that these treatments are an additional burden for the patient. Healthy parts of the organism are also damaged since the commonly used cytostatic agents are unselective and the radiation can have an effect on the entire organism.

The object of the present invention was to improve the tolerability of surgical interventions for malignant tumours and the chemotherapy or/and radiation therapy of malignant tumours.

This object is surprisingly achieved by using a certain combination of ascorbic acid, tocopherol and retinol containing ascorbic acid in a free form and tocopherol and retinol in a free form or/and in the form of pharmaceutically acceptable esters thereof and in a mass ratio relative to one another of 1.5-2.5:0.3-0.8:0.028-0.033 based on ascorbic acid, α-tocopherol as an acetate and retinol as an acetate.

Furthermore the present invention provides a process for producing an agent which improves the tolerability of the chemotherapy or/and radiation therapy of malignant tumours and is characterized in that a combination of ascorbic acid, tocopherol and retinol in which ascorbic acid is present in a free form and tocopherol and retinol are present in a free form or/and in the form of pharmaceutically acceptable esters thereof and are formulated as an active ingredient in a mass ratio relative to one another of 1.5-2.5:0.3-0.8:0.028-0.033 based on ascorbic acid, α-tocopherol as an acetate and retinol as an acetate.

In addition the present invention encompasses a method for improving the tolerability of the chemotherapy or/and radiation therapy of malignant tumours which is characterized in that an agent is administered which contains a combination of ascorbic acid, tocopherol and retinol in which ascorbic acid is present in a free form and tocopherol and retinol are present in a free form or/and in the form of pharmaceutically acceptable esters thereof and they are present in a mass ratio relative to one another of 1.5-2.5:0.3-0.8:0.028-0.033 based on ascorbic acid, α-tocopherol as an acetate and retinol as an acetate.

Furthermore the invention concerns a combination of ascorbic acid, tocopherol and retinol containing ascorbic acid in a free form and tocopherol and retinol in a free form or/and in the form of pharmaceutically acceptable esters thereof, in a mass ratio relative to one another of 1.5-2.5:0.3-0.8:0.028-0.033 based on ascorbic acid, α-tocopherol as an acetate and retinal as an acetate for use as an agent for improving the tolerability of the chemotherapy or/and radiation therapy of malignant tumours.

The agent contains ascorbic acid, tocopherol and retinol in a mass ratio relative to one another of 1.5-2.5:0.3-0.8:0.028-0.033, preferably of 1.6-2.3:0.4-0.68:0.029-0.032, more preferably at 1.8-2.1:0.5-0.65:0.030-0.032, particularly preferably of 2:0.6:0.033 based on ascorbic acid, α-tocopherol as an acetate and retinol as an acetate.

The minimum dose of ascorbic acid, tocopherol and retinol is generally 150 mg/day, 50 mg/day and 3 mg/day, respectively, based on ascorbic acid, α-tocopherol as an acetate and retinol as an acetate. Preferably 1.5 g/day, 300 mg/day and 28 mg/day, respectively, are administered. While maintaining the inventive ratio of the vitamins relative to one another, the dose can be increased but the upper limits of the dose are generally 2.5 g/day, 800 mg/day and 33 mg/day, respectively, based on ascorbic acid, α-tocopherol as an acetate and retinol as an acetate.

Furthermore, according to the present invention it is possible to add β-carotene to the vitamin combination according to the invention as a result of which the amount of retinol used can be reduced. This may be advantageous in order to increase the long-term tolerability of the vitamin combination according to the invention in the patient.

Hence another aspect of the present invention is the use of a combination of ascorbic acid, tocopherol, retinol and β-carotene containing ascorbic acid in a free form and tocopherol and retinol in a free form or/and in the form of pharmaceutically acceptable esters thereof and β-carotene in a free form, and in a mass ratio relative to one another of 1.5-2.5:0.3-0.8:0.028-0.033 based on ascorbic acid, α-tocopherol as an acetate and retinol as an acetate together with β-carotene as an agent for improving the tolerability of the chemotherapy or/and radiation therapy of malignant tumours.

Yet a further aspect of the present invention is a combination of ascorbic acid, tocopherol, retinol and β-carotene containing ascorbic acid in a free form and tocopherol and retinol in a free form or/and in the form of pharmaceutically acceptable esters thereof and β-carotene in a free form and in a mass ratio relative to one another of 1.5-2.5:0.3-0.8:0.028-0.033 based on ascorbic acid, α-tocopherol as an acetate and retinol as an acetate together with β-carotene for use as an agent to improve the tolerability of the chemotherapy or/and radiation therapy of malignant tumours.

According to the invention the total amount of retinol and β-carotene is preferably composed of ⅓ retinol and ⅔ β-carotene where the inventive mass fraction of retinol in the vitamin combination in this aspect of the invention is adhered to the sum of the retinol and β-carotene fractions. The daily dose of β-carotene administered with the vitamin combination according to the invention is preferably 10 to 25 mg, more preferably 15 to 20 mg, particularly preferably 16 to 19 mg. The administered doses of ascorbic acid, tocopherol and retinol are preferably in the ranges defined above.

Ascorbic acid (vitamin C) is a water-soluble vitamin which is contained especially in fresh fruits and vegetables such as rose hip, oranges, lemons and peppers. A vitamin C deficiency (hypovitaminosis C) causes scurvy (bleeding of the skin and gums even to the extent of teeth falling out), susceptibility to infection and growth disorders in children. Ascorbic acid is for example used as an antioxidant in foods where the antioxidant effect is mainly due to the fact that ascorbic acid can act as a radical trap.

Tocopherols (vitamin E) are fat-soluble vitamins which can for example be stored in liver and fat tissue. Of the various tocopherols such as α- β-, γ- and δ-tocopherol, α-tocopherol (including its esters) is usually referred to as the actual vitamin E. Vitamin E has an effect on arachidonic acid metabolism (inhibition of thromboxane biosynthesis) and has a anti-inflammatory effect. Vitamin E appears to mainly act as an antioxidant for unsaturated fatty acids, vitamin A and carotenes by scavenging hyperoxides and peroxides. Increased lipid peroxidation can occur in vitamin E deficiency and muscle weakness and neurodegenerative changes may be found.

Retinol (vitamin A) and esters thereof are also fat-soluble and play an important role in the synthesis of visual purple (rhodopsin) in the retina. Hence a deficiency in this vitamin can lead to damage to the conjunctiva and corneal membrane of the eyes. Furthermore, vitamin A deficiency can trigger growth disorders and an increased susceptibility to disease. Vitamin A has antioxidant properties, but an excessive ingestion of vitamin A can result in disease symptoms such as headache and nausea and also to hair loss, swelling of the bones etc.

β-Carotene is the actual provitamin A and is the most important of the carotenes that belong to the carotinoid group. It occurs in carrots and other vegetables and is used among others as an antioxidant and dye.

The vitamin combination according to the invention can additionally include common pharmaceutical additives such as binders, flavourings, dyes or/and sweeteners.

In the vitamin combination L-(+)-ascorbic acid (vitamin C) can be used in a free form, tocopherol and retinol can be used in a free form and also in the form of their pharmaceutically acceptable esters. β-Carotene is optionally used in a free form. Tocopherol or/and retinol are preferably used in the form of their pharmaceutically acceptable esters e.g. as acetic acid and succinic acid esters; the corresponding acetates are particularly preferably used.

Tocopherol (vitamin E) can be present in the vitamin combination as α-, β-, γ- or/and δ-tocopherol where preferably α- or/and γ-tocopherol and particularly preferably α-tocopherol is used.

The use of the vitamin C, E, A combination according to the invention for antitumour treatment such as in chemotherapy or/and radiation therapy can improve the tolerability and thus the effectiveness of such treatments.

It surprisingly turned out that the ratio of the vitamins relative to one another is crucial for the effect of the combination. It was also found that the daily dose of the respective vitamins (and optionally provitamins) should advantageously be in a certain range (see above). In this connection a so-called no-effect-level was determined below which efficacy was in general no longer observed. The values for vitamins C, E and A in the vitamin combination according to the invention are about 150 mg/day, 50 mg/day and 3 mg/day, respectively, for this (see above).

The vitamins should be taken simultaneously, partially simultaneously or one after the other with a time delay that is not all that great. The vitamins can be administered or taken as tablets, capsules, powder or in an otherwise common form of administration at the appropriate dosage. One variant of this is to use a tablet or capsule which contains ascorbic acid, tocopherol and retinol in the appropriate mass ratio where the core of the tablet or capsule contains the fat-soluble vitamins E and A and this core is surrounded by a layer which contains the water-soluble vitamin C. The vitamin agent can be taken or administered orally or/and parenterally.

Furthermore, the invention envisages an administration by means of injection, in particular intramuscularly, subcutaneously or/and intravenously. In a preferred embodiment the vitamin combination according to the invention is administered intramuscularly which may be particularly advantageous compared to oral administration in patients who can (temporarily) only be fed parenterally. In this connection it is also preferable to administer vitamins E and A (optionally plus provitamin A) intramuscularly but to preferably administer the water-soluble vitamin C subcutaneously or intravenously.

In order to protect healthy tissue and to increase the effectiveness of the corresponding antitumour therapy, the vitamin combination can, according to the invention, be administered before, during or/and after such a therapy.

Use of the agent according to the invention results in a selective protection of healthy tissue which does not impair treatment of the malignant tumour. In contrast an isolated administration of the individual vitamin components contained in the vitamin combination used according to the invention does not result in a selective protection in antitumour therapies. For example it was shown based on animal experiments that an isolated administration of vitamin E results in an accumulation of this vitamin in the tumour since the tumour is a vitamin E trap. FIG. 1 compares the tocopherol contents in certain organs of rats with sarcoma 45.

Accumulation of the antioxidant vitamin E in the tumour of rats increases the resistance, i.e. e.g. the radiation resistance, of the tumour. However, the organs that are not affected by the tumour have such a low vitamin E content compared to sarcoma 45 that they cannot be adequately protected from radiation or cytostatic agents.

In contrast the use of the special vitamin combination according to the invention surprisingly selectively protects healthy tissue, i.e. the toxic manifestations of radiation therapy and cytostatic agents are reduced. This considerably increases the antitumour efficacy of cytostatic agents and irradiation which improves the results of chemotherapy and irradiation in patients with malignant tumours.

Another advantage of the use of the vitamin combination according to the invention is that, due to the reduced side-effects of antitumour therapies, intensive irradiation and chemotherapies are possible which would not have been tolerated by the patients without the protection according to the invention.

When chemotherapies or/and radiation therapies are carried out with the inventive protection from side-effects, substantially fewer local recurrences and distant metastases occur in the affected patients compared to patients which were not protected according to the invention. The 5-year survival rate and the average life-span of such patients is surprisingly considerably increased by the inventive administration of the vitamin C, E, A combination. Hence as a result of the invention patients with malignant tumours can have a long-lasting protection against the toxic effects of antitumour treatment.

In contrast to conventional radiation protective agents such as mexamine, the vitamin agent according to the invention is non-toxic. In addition to its enormous toxicity, mexamine has the disadvantage that it does not have a long lasting radiation-protective effect since it has a short duration of action.

Yet another advantage of the use according to the invention of the vitamin combination is that the vitamin balance of the tumour patients is regulated in a long lasting manner. Normally hypovitaminoses occur in tumour patients even after the malignant tumour has been successfully surgically removed. A normalization of the vitamin balance leads to a stabilization of the immune system of the patient which in turn increases the chances of recovery and thus the average life expectancy.

In addition the invention is characterized by the good availability of the active substances and their simple handling.

It is intended to further elucidate the invention by the figures and examples.

### DESCRIPTION OF THE FIGURES

FIG. 1: Comparison of tocopherol contents in certain organs of rats with sarcoma 45.

FIG. 2: MDA content in rats with sarcoma 45 compared to healthy animals with and without administration of the combination according to the invention.

FIG. 3: Weights of Kroker sarcomas in animals in various experimental groups:
Group I: administration of the vitamin combination according to the invention before cyclophosphamide administration
Group II: only cyclosphosphamide administration
Group III: only the inventive vitamin combination
Group IV: placebo.

FIG. 4: Incidence of the formation of distant metastases in the various groups I-IV (see also table B4.2).

FIG. 5: Graphic representation of the average life-span of patients from the various groups (in months).

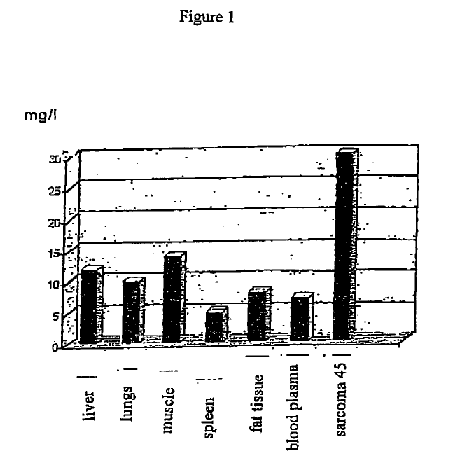

### EXPERIMENTAL AND CLINICAL INVESTIGATIONS

A. Experimental Investigations

1. Dependency of Efficacy on the Dose

The efficacy of the combination of vitamin C, E and A depends on the dose. Experiments on this were carried out on three-month old white male and female rats weighing 150 to 180 g which carried a transplanted sarcoma 45 at the stage of intensive tumour proliferation. The vitamin combination according to the invention was administered to them (group Ia) within 4 to 5 days before their decapitation. Group Ib consisted of comparable rats with transplanted sarcoma 45 which were not administered the vitamin combination. Group Ic which consisted of healthy animals served as an additional comparison.

Another experimental group of rats (IIa) was administered the combination of vitamins C, E and A like group Ia but the dosage was reduced ten-fold. Groups Ib and IIc are again rats with sarcoma 45 or healthy animals, respectively, to which the vitamin combination was not administered.

The doses used are summarized in table A1.1 in mg per gram body weight. The vitamins were injected subcutaneously into the animals.

TABLE A1.1

| | | α-tocopherol | |
|---|---|---|---|
| group | ascorbic acid | acetate | retinol acetate |
| Ia (sarcoma 45) | 0.2-0.3 mg | 0.08-0.09 mg | 0.001-0.002 mg |
| Ib (sarcoma 45) | — | — | — |
| Ic (healthy) | — | — | — |
| IIa (sarcoma 45) | 0.02-0.03 mg | 0.008-0.009 mg | 0.0001-0.002 mg |
| IIb (sarcoma 45) | — | — | — |
| IIc (healthy) | — | — | — |

The animals were killed and the malonic dialdehyde (MDA) concentration in the livers of the animals was examined. Malonic dialdehyde is a product of lipid peroxidation which is increased by tumour proliferation. The concentration of MDA in the liver of the examined group indicates the extent of lipid peroxidation in each case.

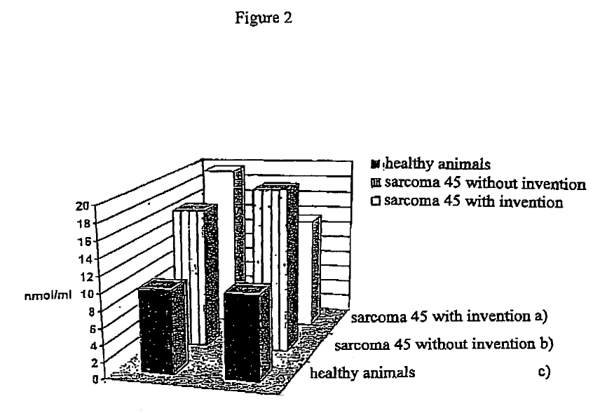

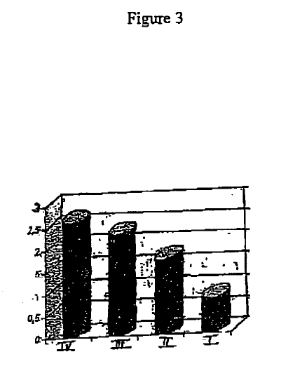

FIG. 2 shows that the MDA concentration in rats with sarcoma 45 is considerably increased compared to healthy animals since there is an increase in the occurrence of lipid peroxidation in these animals. A reduction of the MDA concentration, i.e. lipid peroxidation, caused by the vitamin C, E, A combination is dose-dependent as shown in FIG. 2. The MDA concentration in the liver of rats of group Ia which were administered the vitamin C, E, A combination is considerably reduced compared to sarcoma 45 rats that did not receive the special vitamin combination (group Ib) and almost as low as the MDA concentration of healthy animals (group Ic). In contrast, no antioxidative effect is achieved with a ten-fold reduced dose of the vitamin C, E, A combination; the MDA concentration in the liver of the animals of group IIa is even slightly increased compared to the value that was found in the liver of sarcoma 45 rats which were not administered the vitamin combination (group IIb).

2. Protection by the Vitamin Combination in Irradiation

Three groups each comprising white raceless male mice weighing 18 to 20 g were administered the vitamin combination according to the invention 24 hours before the corresponding whole body irradiation with the gamma ray device “Rokus” (cobalt-60). Vitamins A and E were administered to the animals in an oily solution by means of an oesophageal tube, vitamin C was injected subcutaneously as an aqueous solution. The dose was (in mg per gram body weight) 0.2-0.3 mg ascorbic acid, 0.08-0.09 α-tocopherol acetate and 0.001-0.002 retinol acetate. The mice were irradiated with 8 Gy (group Ia), 12 Gy (group Ib) and 14 Gy (group Ic) and the survival times of the animals was observed. As a comparison three groups of corresponding mice were irradiated but they were not administered the vitamin combination (groups IIa, IIb and IIc).

The results of the irradiation are summarized in table A2.1.

TABLE A2.1

| experimental | % dead animals/days at irradiation dose | | |
|---|---|---|---|
| group | 8 Gy (a) | 12 Gy (b) | 14 Gy (c) |
| I (protection | 16/30 | 50/9 | 50/8 |
| according to the | | | |
| invention) | | | |
| II (without | 26/30 | 50/7 | 50/5 |
| protection) | | | |

Table A2.1 shows that only 16% of the animals from group I were dead 30 days after irradiation with a dose of 8 Gy whereas 26% of the animals of group II which had not been administered the vitamin combination were dead at the same time. When the radiation dose was increased to 12 Gy, 50% of the animals from group I were dead after 9 days and 50% of the animals from group II were already dead after 7 days. The protective effect of the vitamin combination is even more apparent when they were irradiated with a dose of 14 Gy: 50% of the animals from group I still lived after 8 days whereas half of the mice in group II were already dead after 5 days.

Accordingly the average life-span of the mice is considerably longer when they are administered the vitamin combination according to the invention before the irradiation (table A2.2).

TABLE A2.2

| | average life-span (days) at a radiation dose of | |
|---|---|---|
| experimental group | 12 Gy (b) | 14 Gy (c) |
| I (protection according to | 9.0 ± 0.6* | 7.9 ± 0.7** |
| the invention) | | |
| II (without protection) | 7.5 ± 0.5 | 5.5 ± 0.3 |

*p < 0.05
**p < 0.01

The mice survived 9 days on average at an irradiation dose of 12 Gy and 7.9 days at 14 Gy when they were administered the vitamin combination. Animals from group II only survived for 7.5 and 5.5 days, respectively.

In order to compare the radiation protection according to the invention with the effect of conventional radiation protective agents white raceless male and female rats weighing from 150 to 180 g (age: 3 months) was subjected to whole body gamma irradiation at a dose of 11.4 Gy, the animals being protected according to the invention by administering the vitamin combination at the above-mentioned dose 24 hours before the irradiation (group I). On the other hand another group of comparable rats was irradiated identically and the conventional radiation protective preparation, mexamine was administered 24 hours before irradiation (group II). A third group of rats was irradiation without administering a radiation protective agent (group III). The survival rate and average life-span of rats from groups I, II and III are summarized in table A2.3.

TABLE A2.3

| | radiation dose 11.4 Gy | |
|---|---|---|
| experimental group | % dead animals/days | average life-span (days) |
| I (protection | 50/8 | 8.4 ± 4* |
| according to the | 100/16 | |
| invention) | | |
| II (mexamine) | 50/8 | 8.2 ± 4* |
| | 100/13 | |
| III (without | 50/5 | 5.4 ± 0.3 |
| protection) | 100/9 | |

Table A2.3 shows that considerable protection age radiation is achieved by the vitamin combination which results in an increase in the average life-span of the animals compared to unprotected irradiation or irradiation when mexamine is administered. The rats of group I have an average life-span of 8.4 days whereas animals of group II to which mexamine was administered instead of the vitamin agent according to the invention only lived on average 8.2 days after the irradiation. The animals lived 5.4 days on average (group III) when no radiation protective agent was administered.

3. Protection by the Vitamin Combination in Chemotherapy

In order to assess the protective effect of the agent according to the invention a group of white raceless rates weighing from 150 to 180 g (age: 3 months) was subcutaneously injected with the vitamin combination (for dose see 2) 24 hours before injection of 5-fluorouracil. 5-Fluorouracil was administered at a dose of 1.8 mg per kg body weight (group I). Another group of rats did not receive the vitamin combination while being treated correspondingly with 5-fluorouracil. The ability of the rats of these two groups to survive is compared in table A3.1.

TABLE A3.1

| | 5-fluorouracil (1.8 mg per kg |
|---|---|
| | body weight) ability to survive |
| | after 8 days [%] |
| experimental group | (p < 0.002) |
| I (protection according to the invention) | 82 ± 4.3% |
| II (without protection) | 61 ± 5.4% |

Table A3.1 shows that after an observation period of 8 days, 82% of the rats from group I but only 61% of the rats from group II (without protection) had survived.

Hence the combination of vitamins C, E and A according to the invention had an outstanding protective effect in radiotherapy as well as in chemotherapy which was unexpected.

4. Protection by the Vitamin Combination in a Combined Radiotherapy/Chemotherapy

A group of white raceless rats weighing from 150 to 180 g were firstly subjected to a whole body irradiation (gamma ray device see under 2) at a dose of 8 Gy and then 5-fluorouracil (dose: 1.8 mg per kg body weight of the rat) was injected, the vitamin combination according to the invention having been administered 24 hours before the irradiation in the dosage stated in section 2 (group I). A second group of rats was treated correspondingly but the animals were not administered the vitamin agent for protection (group II). The ability of the animals from both groups to survive is compared in table A4.1.

TABLE A4.1

| | irradiation (8 Gy) + 5-fluoruracil |
|---|---|
| | (1.8 mg per kg body weight) |
| | ability to survive |
| experimental group | after 60 days [%] |
| I (protection according to the invention) | 73 |
| II (without protection) | 41 |

Table A4.1 shows that even in the case of a combined chemo/radiotherapy the ability of the experimental animals to survive is vastly increased by the vitamin C, E, A combination according to the invention. Thus 73% of the animals of group I still survived after 60 days whereas only 41% of the animals from group II still live after 60 days.

5. Selectivity of Vitamin C, E, A Combination

The radiation protection and the protection in chemotherapy of the vitamin combination according to the invention acts selectively on healthy tissue and does not reduce the effect of the irradiation or of the cytostatic agent on the tumour. This selectivity was confirmed experimentally by subjecting 4 rats of the above-mentioned species with Walker carcino-sarcoma to a single local irradiation in which only the tumour was irradiated at a dose of 20 Gy using the X-ray device RUM-11. The vitamin combination (dose see under 2.) was administered subcutaneously to two of these rats 24 hours before the irradiation. The tumours of the irradiated donors were transplanted into healthy animals directly after the irradiation. The recipient rats were not administered the vitamin agent. The tumour volumes of all examined animal groups were determined. The results show that there were no differences in the changes of the tumour volumes. Hence the vitamin combination according to the invention had no protective effect against radiation with regard to the malignant tumour and can be used selectively to protect healthy tissue from irradiation.

The selectivity of the agent according to the invention shown in the above experiment was also confirmed by the following investigation:

White raceless mice weighing 20 to 25 g were implanted a Kroker sarcoma by conventional methods. The vitamin combination was administered to one group of the experimental animals 24 hours before they received cyclophosphamide (50 mg per kg body weight) (group I). For comparison a group of mice was treated correspondingly, but without the administration of the vitamin agent according to the invention (group II). The animal group III was only administered the vitamin combination, the control group IV received a placebo. FIG. 3 compares the weight of the Kroker sarcomas of the animals of the various experimental groups. The vitamins were administered at a dose of 300 mg (vitamin C), 90 mg (vitamin E) and 20 mg (vitamin A) in each case per kg body weight.

The results are shown in FIG. 3. This shows that administration of the vitamin agent according to the invention does not protect the tumour against the cytostatic agent cyclophosphamide. This is apparent since the weight of the tumour is even less then when cyclophosphamide is administered alone. The administration of the vitamin combination according to the invention without the administration of a cytostatic agent also results in a reduction of the tumour weight compared to the administration of a placebo.

B. Clinical Investigations

1. Characterization of the Patients

If not stated otherwise in the investigations, the ratio of men to women in the respective treatment groups was practically equal and most patients were aged between 51 and 60 years. They had a body weight of 50-100 kg.

2. Protection by the Vitamin Combination in Surgical Interventions

A group of patients with stomach cancer at an early stage were administered the vitamin C, E, A combination daily in the pre-operative phase for 7 days. Afterwards a surgical intervention was carried out and the operated patients received the vitamin combination over a period of 1.5 years (group I). Another group of comparable patients with stomach cancer at an early stage were operated in a corresponding manner but did not receive the vitamin combination according to the invention (group II).

The vitamin combination was administered orally at a dose of 2.0 g ascorbic acid, 0.6 α-tocopherol acetate and 0.033 g retinol acetate.

The percentage survival rates of the patients of both groups after 1, 3 and 5 years are shown in table B2.1.

TABLE B2.1

| patient group | survival rate (%) after | | |
|---|---|---|---|
| (stage 1B) | 1 year | 3 years | 5 years |
| I (according to the | 96.6 ± 3.2 | 96.6 ± 3.2 | 90.9 ± 6.3* |
| invention) | | | |
| II (only operation) | 90.0 ± 4.7 | 77.5 ± 6.6 | 70.0 ± 6.8 |

*p < 0.025 compared to “only operation”

Table B2.1 shows that the survival rate compared to group II was increased by the administration of the C, E, A combination. After 1 year 5% more patients from group I survive than patients from group II, after 3 years the difference is already almost 20% and after 5 years it is over 20%.

Even in patients with stomach cancer at an advanced stage it was possible to achieve astonishing survival rates in patients by administering the vitamin combination according to the invention. This becomes clear by comparing the percentage survival rates of the patient from group I which in addition to the operation had been administered the vitamin combination, with patients of group II who were only operated on. Table B2.2 compares the survival rates after 1, 3 and 5 years.

TABLE B2.2

| patient group | survival rate (%) after | | |
|---|---|---|---|
| (stage 3A) | 1 year | 3 years | 5 years |
| I (according to the | 85.7 ± 9.3 | 53.0 ± 14.0 | 53.0 ± 14.0* |
| invention) | | | |
| II (only operation) | 84.7 ± 8.1 | 23.5 ± 6.2 | 11.7 ± 5.5 |

*p < 0.01 compared to “only operation”

Especially after 3 and 5 years the survival rate of patients from group I is considerably increased compared to patients from group II; after 3 years by 30%, after5 years by over 40%.

3. Protection by the Vitamin Combination in Chemotherapy

a)

A group I comprising patients with stomach cancer at stage IV (13 patients) was intravenously administered 750 mg 5-fluorouracil daily over 7 days. The pauses between the treatment cycles lasted 2 to 2.5 months. These patients were administered the vitamin combination daily during the entire chemotherapy at a daily dose as stated in section 2. A corresponding therapy with 5-fluorouracil was carried out with a group II comprising patients at the same stage of the disease but without protection by the vitamin agent.

The results of the control groups are taken from older literature references.

Table B3.1 compares the survival rates of patients from groups I and II.

TABLE B3.1

| patient group | survival rate (%) after (months) | | | | | | |
|---|---|---|---|---|---|---|---|
| (stage IV) | 3 | 6 | 9 | 12 | 18 | 22 | 44 |
| I 5-Fu + invention) | 100 | 77.0 | 53.9 | 46.2 | 30.8 | 30.8 | 23.1 |
| II (only 5-Fu) | 86.7 | 66.7 | 40.0 | 20.0 | 13.7 | 0 | — |

Table B3.1 shows that the survival rate of the patients with stomach cancer at stage IV is considerably increased when they are administered the vitamin combination during the chemotherapy. After 44 months 23.1% of the patients from group I are still alive. However, in group II no patient survived up to the 22nd month. The survival rate of group I after 12 and 18 months is more than twice as high as that of group II. Hence the invention makes it possible to considerably increase the effectiveness of chemotherapy and thus to ensure a longer survival of the patients.

The results of the group studies that were carried out show that the vitamin combination of the invention increases the antitumour effectiveness of chemotherapies and considerably reduces their toxic effect in the organism which improves the results of chemotherapy in patients with stomach cancer at an advanced stage.

b)

A gastric carcinoma with initial skin metastasis was diagnosed in June 2002 in a 74 year old female patient. Histologically it was a less differentiated adeno-carcinoma matching the diffuse type according to Lauren. A laparotomy was performed in August 2002, a peritoneal carcinosis was found but the finding was inoperable. Only biopsy specimens were taken and a port system was implanted to administer chemotherapy. 5-Fu was continuously infused for 5 days in a total of 4 cycles at 4 week intervals; the infused dose was 600 mg/m² (BS) body surface daily. This was followed by administration of 2 further cycles at a dose of 750 mg/m² BS daily also for 5 days in each case. During the entire chemotherapy period, the vitamin complex was daily ingested orally at the following dosage: vitamin C 2000 mg, vitamin E 600 mg and vitamin A 33 mg. The health of the patient was excellent during this period neither decreases in leucocytes nor nausea, vomiting or mucositis were observed. Imaging and blood chemistry control examinations showed that the disease was absolutely stable under the combined treatment of 5-FU and the vitamin combination according to the invention.

This treatment result shows that tolerance to chemotherapy is considerably increased by the vitamin combination according to the invention and at the same time an improved therapeutic result can be observed. Hence the present invention increases the chances of a successful treatment and thus improves the life expectancy of the affected patients even in the case of severe cancer diseases.

4. Protection by the Vitamin Combination in Radiation Therapies

A major problem in the case of tumour patients is that these patients usually suffer from a relapse in the post-operative phase and distant metastases form. This occurs in 20 to 50% of the patients.

Patients with adeno-carcinoma at different stages were subjected to various treatments. Group I was exposed to radiotherapy at a dose of 20 Gy in the pre-operative phase during which the patients were administered the vitamin combination according to the invention. This was followed by the operation. Patients in group II were treated like patients from group I but the radiation dose was 30 Gy. Patients from group III were subjected to radiotherapy at a dose of 20 Gy before the operation but they did not receive the vitamin combination. Group IV consisted of patients which were only operated on.

The incidence of the formation of distant metastases in patients from groups I to IV is shown in table B4.1 versus their adeno-carcinoma structure after an observation period of 5 years.

TABLE B4.1

| adeno- | group I | | group II | | group III | | group IV | |
|---|---|---|---|---|---|---|---|---|
| carcinoma | A* | | A* | | A* | | A* | |
| stage | B | in % | B | in % | B | in % | B | in % |
| highly | 0 | 0.0 | 0 | 0.0 | 2 | 33.3 | 6 | 18.8 |
| differentiated | 1 | | \overline{11} | | 6 | | \overline{32} | |
| moderately | 5 | 26.3 | 2 | 7.7 | 2 | 20.0 | 9 | 20.9 |
| differentiated | \overline{19} | | \overline{26} | | \overline{10} | | \overline{43} | |
| less | 0 | 0.0 | 0 | 0.0 | 2 | 100 | 0 | 0.0 |
| differentiated | 3 | | 4 | | 2 | | 9 | |

*A = number of patients with distant metastases
B = total number of patients

The formation of distant metastases of a highly differentiated adeno-carcinoma was registered most frequently in group III: 33.3% of the patients in this group had distant metastases after an observation period of 5 years. This value is much higher than in the patients which were only subjected to an operation (group IV, 18.8%). Hence the radiation therapy had a damaging effect on the organism of the patients unless the patients are administered the vitamin combination according to the invention for protection. Thus in patients of group I which were also irradiated with 20 Gy but were protected by the vitamin combination according to the invention, no distant metastases were found. Even when the radiation was increased to 30 Gy (group II), the patients had no distant metastases. This effect is pronounced even in the case of less differentiated adeno-carcinomas: all patients, i.e. 100% of group III, had distant metastases whereas distant metastases were observed neither in group I nor in group II:

The protective effect of the vitamin agent in various radiation therapies can even be observed in patients with colon cancer at various stages. The incidence of distant metastases formation in patients with colon cancer at various stages after an observation period of 5 years is listed in table B4.2. Patients of groups I to IV were subjected to the above-mentioned therapies.

TABLE B4.2

| colon | group I | | group II | | group III | | group IV | |
|---|---|---|---|---|---|---|---|---|
| cancer | A* | | A* | | A* | | A* | |
| stage | B | in % | B | in % | B | in % | B | in % |
| T3-4 NO-MO (II) | 2 | 12.5 | 1 | 5.0 | 2 | 18.2 | 8 | 14.5 |
| | \overline{16} | | \overline{20} | | \overline{11} | | \overline{55} | |
| T2-4N1-MO (III) | 2 | 28.6 | 1 | 8.3 | 4 | 50.0 | 8 | 47.1 |
| | 7 | | \overline{12} | | 8 | | \overline{17} | |

*A = number of patients with distant metastases
B = total number of patients

It is particularly apparent from table B4.2 that the vitamin combination according to the invention increases the effectiveness of radiotherapy since the formation of distant metastases in patients of groups I and II is reduced in comparison to patients that are only irradiated but not protected according to the invention. Hence the radiation dose can be increased without damaging healthy tissue of the patients. Patients of group II which were irradiated with 30 Gy had only 5% (T3-4NO-MO (II)) or 8.3% (T2-4N1-MO(III)) distant metastases whereas patients of group III who had only been irradiated with 20 Gy and had received no vitamin combination had 18.2% and 50.0% distant metastases, respectively.

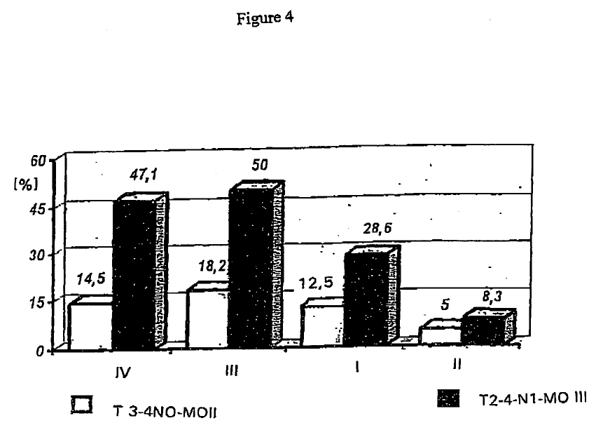

These results are shown graphically in FIG. 4.

The overall incidence of distant metastasis formation depending on the treatment method (groups I to IV) in patients with colon cancer after an observation period of 5 years is shown in table B4.3.

TABLE B4.3

| | group I | | group II | | group III | | group IV | |
|---|---|---|---|---|---|---|---|---|
| | A* | | A* | | A* | | A | |
| | B | in % | B | in % | B | in % | B | in % |
| number of | 5 | 21.7 ± 1.9 | 2 | 4.9 ± | 6 | 30.0 ± 4.5 | 16 | 18.4 ± 3.4 |
| patients | \overline{23} | | \overline{41} | 1.5** | \overline{20} | | 87 | |
| with | | | | | | | | |
| distant | | | | | | | | |
| metastases | | | | | | | | |
| formation | | | | | | | | |

*A = number of patients with distant metastases
B = total number of patients
**p < 0.05 compared to group III

The formation of distant metastases occurs most frequently when patients are irradiated (30%, group III). The vitamin combination according to the invention can effectively protect healthy tissue from radiation. Only 21.7% of patients from group I which were irradiated correspondingly under the inventive protection had distant metastases. The protective effect is particularly evident since this value is even substantially lower (4.9%) at a higher radiation dose (30 Gy, group II).

The use of the vitamin combination in pre-operative radiation therapy results in a considerable reduction in the damage to healthy tissue. The protection according to the invention enables one to irradiate at higher doses and thus to increase the effectiveness of therapies.

5. Protection by the Vitamin Combination in Combined Chemo/Radiotherapy

a)

Various patient groups were subjected to a combined chemo/radiotherapy. Patients with pancreatic cancer were treated surgically and then subjected to a postoperative radiation therapy with subsequent polychemotherapy.:

| | |
|---|---|
| group I: | Radiation therapy with a total focal dose of 35 Gy |
| | (single focal dose 7 Gy), 6 treatment cycles |
| | of polychemotherapy. |
| group II: | Radiation therapy as a secretion cure at |
| | radiation doses of 28 Gy (single |
| | focal dose 4 Gy) plus 30 Gy (single |
| | focal dose 2 Gy), 6 treatment cycles |
| | of polychemotherapy. |

The polychemotherapy was carried out 15 to 20 days after the radiation therapy. 300 mg/m² 5-fluorouracil was administered within the first 5 days, 50 mg/m² doxorubicin was administered on the first day and 20 mg/m² cis-platinum was administered during the first 5 days. The cytostatic agents were administered intravenously. Pauses between the treatment cycles of the polychemotherapy were 25 to 30 days. All patients of groups I and II were given the vitamin combination orally, once per day 24 hours before each treatment. A group of patients which had only been treated surgically served as a comparative group (group III). The data shown for the control group III are derived from experiments cited in older literature references.

The survival rates of patients from groups I to III are compared in table B5.1

TABLE B5.1

survival rate (%) after

| patient group | 1 year | 2 years | 3 years | 5 years |
|---|---|---|---|---|
| I | 30.0 | 5.0 | 0 | — |
| II | 45.0 | 15.0 | 10.0 | 5.0 |
| III | 0 | — | — | — |
| (control) | | | | |

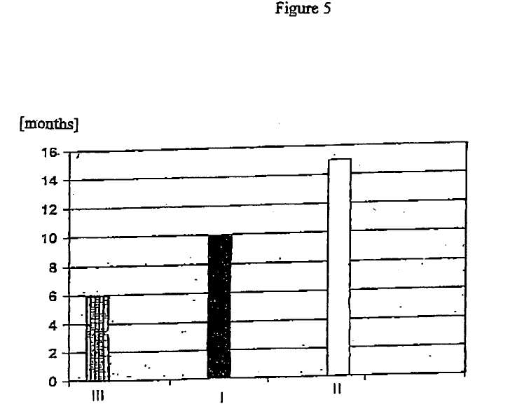

All patients in the control group III died within 12 months. In contrast, combined chemo/radiotherapy together with the vitamin agent enable 5% of patients in group II to still live even after 5 years. The average survival time of the patients of the three study groups is shown graphically in FIG. 5.

A comparison with a group which had been subjected to the same chemo/radiotherapy as groups I and II but without protection by the vitamin agent is ethically unacceptable but the values may be taken from control groups in the literature. They show that the toxicity of the administered cytostatic agents and the radiation doses are so severe that the effectiveness of the treatments is limited. Only with the protection of the vitamin combination according to the invention is it possible to use such high doses which makes the therapies extremely successful.

b)

An inflammatory mammary carcinoma of the left breast was diagnosed in April 2002 in a 59 year old female patient. The histological findings were a less differentiated invasive ductal mammary carcinoma with extensive lymphangiosis carcinomatosis of the skin. Hormone receptor status: oestrogen receptor 60%, progesterone receptor 70% positive, Her2-new: dako score 1-2, hence borderline. 6 cycles of chemotherapy with epirubicin/cyclophosphamide were administered intravenously on day 1 at 3-week intervals as a neoadjuvant. 4 cycles the dose was 90 mg/m² BS epirubicin, 600 mg/m² BS cyclophosphamide. In the last 2 cycles the epirubicin dose was increased to 120 mg/m². From September to October 2002 the left breast and the associated lymphatic drainage tracts were irradiated at 50 Gy. During the irradiation the patient received the vitamin combination according to the invention daily which comprised 2000 mg vitamin C, 600 mg vitamin E and 33 mg vitamin A. The radiation therapy was tolerated very well and there was only a slight reddening of the irradiated skin areas; nausea and vomiting occurred just as little as decreases in leukocytes. Punch specimens were taken from the left breast in December 2002 which gave no indication of malignancy.

This treatment report is a further impressive illustration of the improvement of the tolerability of antitumour therapy due to the administration of the vitamin combination according to the invention. It should be noted in particular that despite a very high irradiation dose of 50 Grey, none of the otherwise common side-effects such as deterioration of the blood picture, nausea etc. were observed. In general such high radiation doses are associated with severe side-effects. Hence administration of the agent according to the invention enables more effective chemo/radiotherapies to be used and thus improves the chances of recovering from malignant tumours. This is for example illustrated by the above-mentioned case in which malignancy was no longer diagnosed in the punch specimens taken from the left breast of the patient after treatment according to the invention.

In summary it may be stated that administration of the vitamin C, E, A combination according to the invention considerably increases the tolerability of radiation or/and chemotherapies since healthy tissue is selectively protected. Thus it is possible to also apply or administer very high radiation doses and very high doses of cytostatic agents.

1. Use of a combination of ascorbic acid, tocopherol and retinol containing ascorbic acid in a free form and tocopherol and retinol in a free form or/and in the form of pharmaceutically acceptable esters thereof in a mass ratio relative to one another of 1.5-2.5:0.3-0.8:0.028-0.033 based on ascorbic acid, tocopherol as an acetate and retinol as an acetate, as an agent for improving the tolerability of the chemotherapy or/and radiation therapy of malignant tumours.

2. Process for producing an agent which improves the tolerability of the chemotherapy or/and radiation therapy of malignant tumours,
characterized in that,
a combination of ascorbic acid, tocopherol and retinol is used in which ascorbic acid in a free form and tocopherol and retinol in a free form or/and in the form of pharmaceutically acceptable esters thereof are formulated as an active ingredient in a mass ratio relative to one another of 1.5-2.5:0.3 0.8:0.028-0.033 based on ascorbic acid, a tocopherol as an acetate and retinol as an acetate.

3. Method for improving the tolerability of the chemotherapy or/and radiation therapy of malignant tumours,
characterized in that,
an agent is administered which contains a combination of ascorbic acid, tocopherol and retinol in which ascorbic acid is present in a free form and tocopherol and retinol are present in a free form or/and m the form of pharmaceutically acceptable esters thereof and they are present in a mass ratio relative to one another of 1.5-2.5:0.3-0.8:0.02-0.033 based on ascorbic acid, a-tocopherol as an acetate and retinol as an acetate.

4. Use as claimed in claim 1,
characterized in that,
the agent contains ascorbic acid, tocopherol and retinol in a mass ratio relative to one another of 1.8-2.1:0.5-0.65:0.030-0.032 based on ascorbic acid, α-tocopherol as an acetate and retinol as an acetate.

5. Method as claimed in claim 2,
characterized in that,
the agent contains ascorbic acid, tocopherol and retinol in a mass ratio relative to one another of 1.8-2.1:0.50. 65:0.030-0.032 based on ascorbic acid, α-tocopherol as an acetate and retinol as an acetate.

6. Use as claimed in claim 1,
characterized in that,
ascorbic acid, tocopherol and retinal are administered at a minimum dose of 1.50 mg/day, 50 mg/day and 3 mg/day and at a maximum dose of 2.5 g/day, 800 mg/day and 33 mg/day each based on ascorbic acid, α-tocopherol as an acetate and retinol as an acetate.

7. Use as claimed in claim 6,
characterized in that,
the minimum dose of 1.5 g/day, 300 mg/day and 28 mg/day based on ascorbic acid, α-tocopherol as an acetate a-ad retinal as an acetate is administered.

8. Use as claimed in claim 1,
characterized in that,
the agent contains α-tocopherol or/and retinal in the form of pharmaceutically acceptable esters thereof.

9. Use as claimed in claim 8,
characterized in that,
the agent contains α-tocophervl or/and retinol in the form of acetic acid or/and succinic acid esters thereof.

10. Use as claimed in claim 8,
characterized in that,
the agent contains α-tocopherol and retinol in the form of their acetates.

11. Use as claimed in claim 1,
characterized in that,
the agent additionally contains pharmaceutically acceptable binding agents, flavourings, dyes, sweeteners or/and other common pharmaceutical additives.

12. Combination of ascorbic acid, tocopherol and retinol in which ascorbic acid is present in a free form and tocopherol and retinol are present in a free form or/and in the form of pharmaceutically acceptable esters thereof. and they are present in a mass ratio relative to one another of 1.5-2.5;0.3-0.8:0.028-0.033 based on ascorbic acid, α-tocopherol as art acetate and retinol as an acetate for use as an agent to improve the tolerability of the chemotherapy or/and radiation therapy of malignant tumours.

13. Use of a combination of ascorbic acid, tocopherol, retinol and β-carotene in which ascorbic acid is present in a free form and tocopherol and retinol are present in a free form or/and in the form of pharmaceutically acceptable esters thereof and β-carotene is present in a free form and they are present in a mass ratio relative to one another of 1.5-2.5:0.3-0.8:0.028-0.033 based on ascorbic acid, α-tocopherol as an acetate and retinol as an acetate together with β-carotene, as art agent to improve the tolerability of the chemotherapy or/and radiation therapy of malignant tumours.

14. Process for producing an agent which improves the tolerability of the chemotherapy or/and radiation therapy of malignant tumours,
characterized in that,
a combination of ascorbic acid, tocopherol, retinol and β-carotene is used in which ascorbic acid in a free form and tocopherol and retinol in a free form or/and in the form of pharmaceutically acceptable esters thereof and β-carotene in a free form are formulated as an active ingredient in a mass ratio relative to one another of X.52.5:0.3-0.8:0.028-0.033 based on ascorbic acid, a tocopherol as an acetate and retinol as an acetate together with β-carotene.

15. Use as claimed in claim 13,
characterized in that,
the total amount of retinol and β-carotene is composed of ⅓ retinal and ⅔ β-carotene.

16. Process as claimed in claim 14,
characterized in that,
the total amount of retinal and β-carotene is composed of ⅓ retinal and ⅔ β-carotene.

17. Use as claimed in claim 13,
characterized in that,
the dose of β-carotene is 10 mg/day to 25 mg/day.

18. Process as claimed in claim 14,
characterized in that,
the dose of β-carotene is 10 mg/day to 25 mg/day,

19. Combination of ascorbic acid, tocopherol, retinol and β-carotene containing ascorbic acid in a free form and tocopherol and retinol in free form or/and in the form of pharmaceutically acceptable esters thereof and β-carotene in a free form in a mass ratio relative to one anther of 1.5-2.5:0.3-0.8:0.0.028-0.033 based on asorbic acid, α-tocopherol as an acetate and retinol as an acetate together with β-carotene for use as an agent to improve the tolerability of the chemotherapy or/and radiation therapy of malignant tumors.
